(19) United States
(12) Patent Application Publication
(10) Pub. No.: US 2011/0230939 A1
(43) Pub. Date: Sep. 22, 2011

(54) System for diagnosing and treatment of diabetic symptoms

(76) Inventor: Ronald J. Weinstock, Fort Lauderdale, FL (US)

(21) Appl. No.: 13/065,305

(22) Filed: Mar. 18, 2011

Related U.S. Application Data
(63) Continuation-in-part of application No. 13/065,015, filed on Mar. 11, 2011.
(60) Provisional application No. 61/340,497, filed on Mar. 18, 2010.

(51) Int. Cl. A61N 1/36

(52) U.S. Cl. 607/65

(57) ABSTRACT

The provision of a system of electrical, electromagnetic or magnetic stimulation to one or more of the T6 through T12 vertebrae of the human spine, through the use of probes, imparts one or more of low frequency, high frequency, AC, DC and combinations, through the sympathetic and parasympathetic nervous systems, to stimulate the activity of beta cells of the human pancreas, to innervate such cells to better approximate normal function, inclusive of enhanced release of insulin from such cells of the pancreas.

### CROSS REFERENCE TO RELATED APPLICATIONS

This application claims the benefit under 35 USC 119(e) of the provisional patent application Ser. No. 61/340,497 filed Mar. 18, 2010, entitled System for Diagnosing and Treatment of Diabetic Symptoms, which is hereby incorporated by reference in its entirety; and is a continuation-in-part of application Ser. No. 13/065,015, filed Mar. 11, 2011, entitled EMF Probe Configurations for Electro-Modulation of Ionic Channels of Cells and Methods of Use Thereof, which is incorporated herewith in its entirety.

### FIELD OF THE INVENTION

The present invention relates to methods for regulating electrical movement of ions useful to the treatment of diabetes.

### BACKGROUND OF THE INVENTION

The role of biological ions as mediators of cellular activity is well established. Various technologies exist for controlling movement of ionic species across the membrane of living cell. Herein, the effectuation of such movement at a distance, using axonic pathways of the nervous system, is explored with specific reference to the spinal cord relative to the pancreas.

Prior art known to the inventor of an electrotherapeutic treatment of diabetes is reflected in U.S. Patent Application Publication U.S. 2004/0249416 to Yun et al, entitled Treatment of Conditions thru Electrical Modulation of the Autonomic Nervous System. The inventors method and system differ in many respect from the work of Yun et al.

### SUMMARY OF THE INVENTION

The present method relates to the provision of electrical, electromagnetic or magnetic stimulation to one or more of the T6 through T12 and related neural off-shoots of these vertebrae of the human spine, through the use of probes, induction coils and electrodes to impart one or more of low frequency, high frequency, AC, DC and combinations thereof, through the sympathetic and parasympathetic nervous systems, to appropriately stimulate the activity of beta cells of the human pancreas, to innervate such cells to better approximate normal function, inclusive of enhanced release of insulin from such cells of the pancreas.

It is accordingly an object of the invention to provide an electrotherapeutic means of treatment of diabetes.

It is another object to enhance activity of beta cells of the human pancreas in order to preclude onset of diabetes-like symptoms.

It is a further object of the invention to monitor selected electromagnetic wave patterns within the T6 to T12 and related neural off-shoots and vertebrae to provide an early diagnosis, or diagnosis of, susceptibility to diabetes.

The above and yet other objects and advantages of the present invention will become apparent from the hereinafter set forth Brief Description of the Drawings and Detailed Description of the Invention.

### BRIEF DESCRIPTION OF THE DRAWINGS

FIG. 1 is a schematic view of the sympathetic and parasympathetic nervous systems and selected internal organs of the human body related thereto.

FIG. 2 is a flow diagram showing cytoplasmic calcium and other changes that occur when membrane potential changes are sensed by a cell.

FIG. 3 is a diagrammatic view showing the role that the Ca2⁺ and K⁺ channels play in insulin secretion.

FIG. 4 is a graph showing the relationship between cell membrane potential, and calcium ion related current flow in a human cell.

FIG. 5 is a graph showing the relationship between cell membrane potential and concentration of free calcium ions within a cell.

FIG. 6 is a three-dimensional graph showing the relationship between cell membrane potential, calcium ion related current flow into the, cell and percent of time that calcium gated channels of the cell are open.

FIGS. 7-9 show diagnostic waveforms applied for cell treatment.

FIGS. 10 and 11 show electrical waveforms associated with a treatment of a first patient.

FIGS. 12-15 show electrical waveforms associated with treatment of a second patient.

FIGS. 16-17 show concepts for imagining of parameters relevant to normalization of cell function.

FIG. 18 is an illustration of preferred locations of electrical pads used in the practice of the present invention in connection with the treatment of diabetes and hypertension-related conditions.

### DETAILED DESCRIPTION OF THE INVENTION

As is well-known, the sympathetic nervous system (SNS) is a branch of the autonomic nervous system and of the central nervous system (CNS) and is related to the parasympathetic nervous system (PNS).

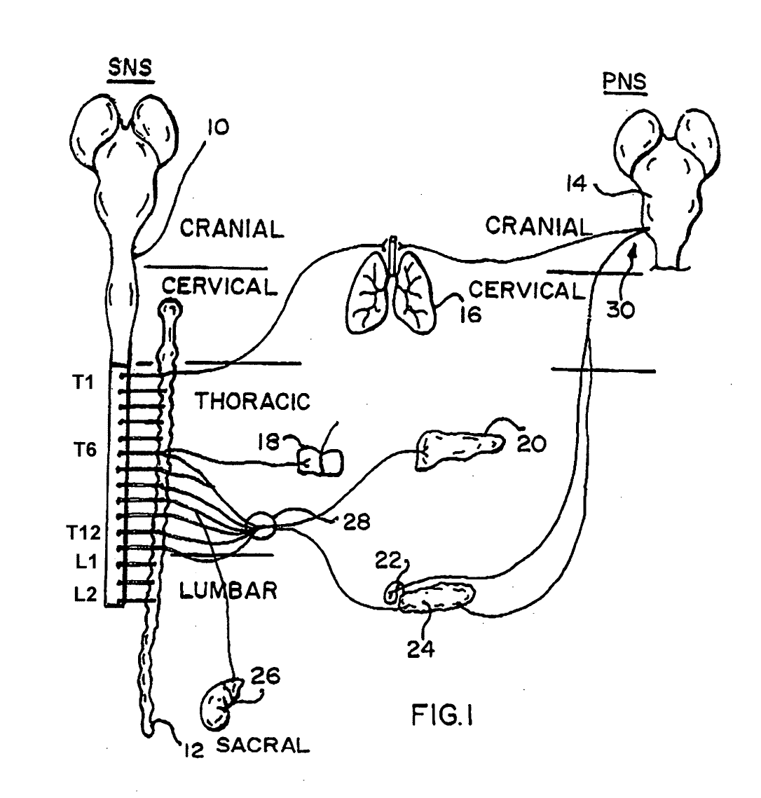

The SNS is active at a so-called basal level and becomes active during times of stress. As such, this stress response is termed the fight-or-flight response. The SNS operates through a series of interconnected neurons. Sympathetic neurons are frequently considered part of the PNS, although many lie within the CNS. Sympathetic neurons of the spinal cord are of course part of the CNS, and communicate with peripheral sympathetic neurons through a series of sympathetic ganglia. For purposes of the present invention, the CNS may be viewed (see FIG. 1) as consisting of a spinal cord 10 and a sympathetic trunk 12 thereof.

The PNS is shown to the right of FIG. 1 as numeral 14. The PNS is considered an automatic regulation system, that is, one that operates without the intervention of conscious thought. As such, fibers of the PNS innervate tissues in almost every organ system, providing at least some regulatory function to areas as diverse as the diameter of the eye, gut motility, and urinary output. For purposes of the present invention, the only organs so regulated by the SNS shown are lung 16, hair follicles 18, liver 20, gall bladder 22, pancreas 24, adrenal glands 26, and hypertension generally. As may be noted in FIG. 1, all neurons of nerves of the SNS of interest originate in the thoracic vertebrae of the spinal cord and pass through sympathetic trunk 12 thereof. This is known as the thoracolumbar outflow of the SNS. Therein, axons of these nerves leave the spinal cord through anterior outlets/routes thereof of the sympathetic trunk 12 and, certain groups thereof, including the groups emanating from thoracic vertebrae T6 through T12 reach celiac ganglion 28 before dispersing to various internal organs in the thoracic region of the body including pancreas 24. From these internal organs occurs a flow of axons of these respective nerves to the base of the PNS at the vagus nerve 30 shown in FIG. 1.

To reach target organs and glands, axons must travel long distances in the body, and to accomplish this, many axons relay their message to a second cell through synaptic transmission. This entails the use of a nuero-transmitter across what is termed the synaptic cleft which activates further cells known as post-synaptic cells. Therefrom, the message is carried to the final destination in the target organ.

Messages travel through the SNS in a bi-directional fashion. That is, so-called efferent messages can trigger changes in different parts of the body simultaneously to further the above referenced fight-or-flight response function of the SNS. It is noted that the PNS, in distinction to the CNS, controls actions that can be summarized as rest-and-digest, as opposed to the fight-or-flight effects of the SNS. Therefore, many functions of the internal organs are controlled by the PNS in that such actions do not require immediate reaction, as do those of the SNS. Included within these is the control of the gall bladder 22 and pancreas 24 by the SNA, as may be noted in FIG. 1.

It may thereby be appreciated that the autonomic nervous system includes both said SNS and PNS divisions which, collectively, regulate the body's visceral organs, their nerves and tissues of various types. The SNS and PNS must, of necessity, operate in tandem to create synergistic effects that are not merely an “on” or “off” function but which can better be described as a continuum of effect depending upon how vigorously each division must execute its function in response to given conditions. The PNS often operates through what are known as parasympathetic ganglia and includes so-called terminal ganglia and intramural ganglia which lie near the organs which they innervate, this inclusive of the pancreas.

In summary, a change of axon activity within an internal organ is measurable at one or more of the T6 through T12 thoracic locations of the SNS and, in principle, also at the vagus nerve 30 of the PNS, above described.

The inventor, in clinical studies, has noticed that a dysfunction of a given internal organ can be recognized by a retardation of signal strength and stability within the neurons at the T6 through T12 locations of the spinal cord. More particularly, in persons suffering from diabetes, I have found weakness and instability of neuro-transmitted signals which would normally pass from pancreas 24, through celiac ganglien 28 and to vertebrae T6 to T12 of the spinal cord. See FIG. 1.

It is believed that appropriate measurements, if taken, at vagus nerve 30 of the PNS would show a similar retardation or instability of otherwise normal signal reaching the cranial base through the nerves of the PNS. Responsive to the above observations, I propose treatment of this instability of the internal organs, inclusive of the pancreas, by the application of appropriate electromagnetic signals through either, or both, the T6 through T12 of the SNS and at the vagus nerve of the PNS, as a means of treating reduced pancreatic function.

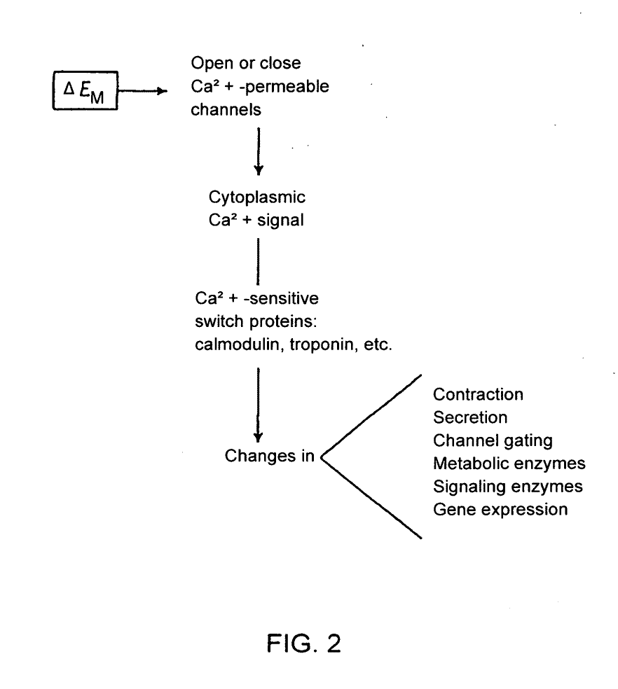

That cells of the human body are acutely responsive to electrical and electromagnetic stimulation through neurotransmitters and otherwise, has long been established by research in the area. Calcium has been determined to be the final transmitter of electrical signals to the cytoplasm of human cells. More particularly, changes in cell membrane potential are sensed by numerous calcium-sensing proteins of cell membrane which determine whether to open or close responsive to a charge carrying elements, in this case, the calcium anion Ca²+. This is shown conceptually in FIG. 2 which shows the electrical call to action of a cell upon its sensing of a voltage gradient carried or created by a calcium anion. Stated otherwise, calcium ions transduce electrical signals to the cells through what are termed voltage-gated calcium channels (see Hille, “Ion Channels of Excitable Membranes,” 3 Ed., 2001, Chap. 4). It is now recognized that electrical signaling of voltage-gated channels (of which there are many categories) of human cell membranes is controlled by intracellular free calcium (and other) ionic concentrations, and that electrical signals are modulated by the flow of calcium anions into cytoplasm from the external medium or from intra cellular stores.

One well-studied calcium dependent process is the secretion of neuro-transmitters at nerve terminals. See Hille, page 104 thereof. Within the presynaptic terminal of every chemical snyapse, there are membrane-bounded vesicular-containing high concentrations of neurotransmitter molecules of various types. When such an action potential engages a neurotransmitter, the membranes having one or more of these vesicules in their surface membrane, release a group of neuro-transmitters into the cellular space. This is conceptually shown in FIG. 2. In the pancreas, there exist so-called pancreatic acinar cells which contain zymogen granules which assist in cellular functions thereof.

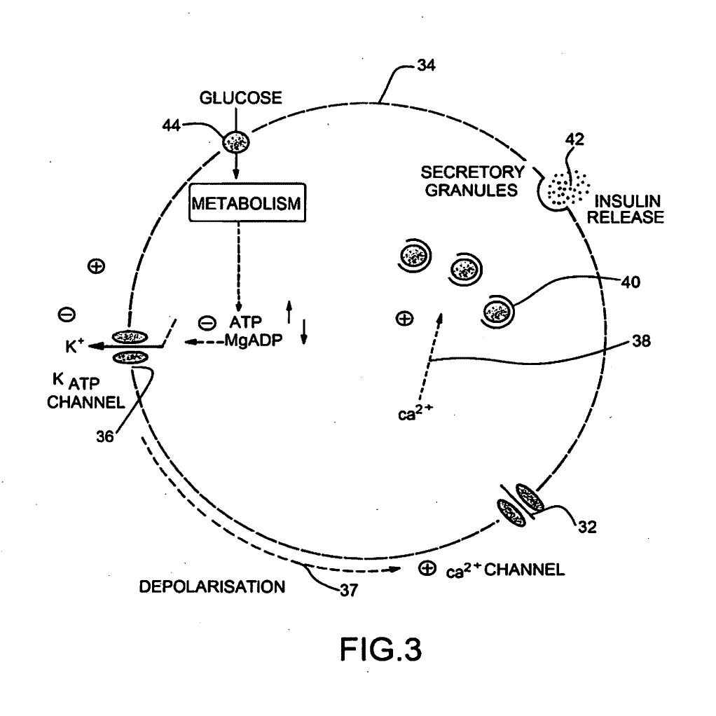

Normally stimulated secretion from nerve terminal of most excitable cells require the extracellular calcium anions Ca²⁺ pass thru ionic channels of the cell. The above is shown at a cellular level in the schematic view of FIG. 3 which shows the calcium ionic channel 32 of cell 34 as well as the egress of a potassium anion through a so-called KATP channel 36 when a calcium anion enters the cell. This process triggers a variety of functions which relate to insulin secretion. Lack of sufficient secretion is of course the primary cause of diabetes as it is broadly understood. FIG. 3 therefore illustrates the current model of insulin secretion (Ashcroft, “Ion Channels and Disease,” 2000, p. 155).

In summary, FIG. 3 indicates that when plasma glucose levels rise, glucose uptake and metabolism by the pancreatic beta cells is enhanced, producing an increase in the intracellular ATP which is a cellular energy source. These changes act in concert to close calcium channels 36 in the beta-cell membrane because ATP inhibits, whereas MgADP (shown in FIG. 3) activates, calcium ion channel activity. In that calcium channel activity determines the beta cell resting potential, its closure causes a membrane depolarization 37 that activates voltage-gated calcium anion channels 32, increasing calcium influx and stimulating insulin release. Insufficient charge upon intracellular calcium may, it is believed, be one cause of inhibition of the above-described normal metabolic process of the pancreatic beta cells. In other words, if intracellular calcium, or its relevant neurotransmitters, lack sufficient charge, insufficient electrical energy 38 is provided to secretory granules 40 sufficient to effect insulin release 42, that is necessary to metabolize glucose 44.

Another view of insulin secretion is that, by blockage of potassium ion channels 36, sufficient charge can be sustained within the cell to maintain normal function of secretory granules 40 and therefore of insulin release 42. Therapeutic drugs which seek to so modulate insulin secretion by control of the potassium channels are sulphonylureaus and diazoxide.

In summary, when blood glucose 44 rises, the uptake thereof is increased by the action of the calcium anions Ca²⁺ entering cell 34. Aspects of this metabolism cause the potassium ATP channels 36 to close which results in membrane polarization 37, a change of voltage potential at calcium ion channels 32, and an increase in cytoplasmic anionic calcium that triggers the function of insulin secretory granules 40. It is therefore desirable to regulate calcium channel activity by maintaining a low level of blood glucose. This requires that an adequate molarity of Ca²⁺ exist in the beta cells.

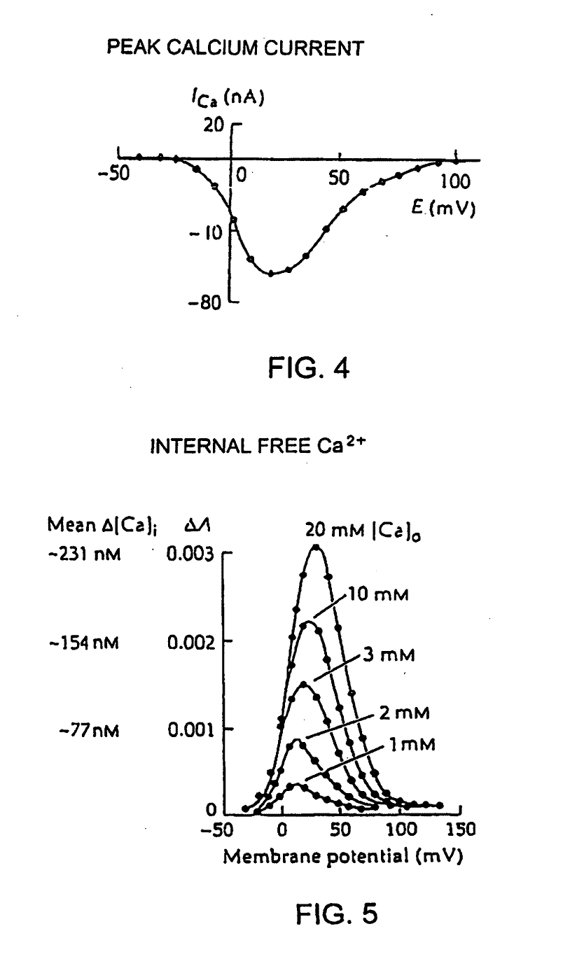

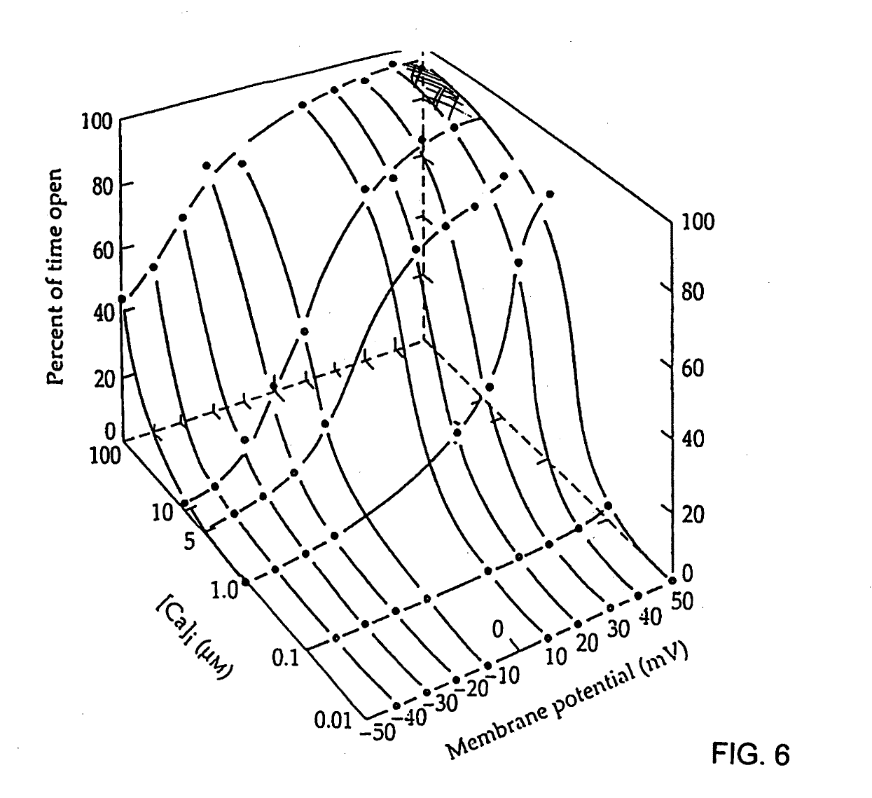

The relation of the offset of ionic calcium on membrane potential of the cell, ionic current flow within the cell, and molarity of calcium within the cell are shown in FIGS. 4 and 5 respectively. FIG. 4 indicates that the percent of time of calcium channel opening as a function of membrane potential and calcium molarity within the intracellular media. Stated otherwise, an increase in membrane potential will increase the time that voltage-gated ionic channels of the cell are open. In view of the above, it appears an appropriate increase in ionic calcium within beta cells of the pancreas will bring about an increase in insulin release if supported by a sufficiency of the membrane potential. The cross-hatched area at the top of the graph of FIG. 6 represents a confluence of parameters most beneficial to the health of the cell.

In view of the above, the inventor proposes the delivery of such enhanced membrane potential to beta cells of the pancreas through the SNS and/or PNS, as above described with reference to FIG. 1, by the application of appropriate electromagnetic signals at the T6 through T12 thoracic vertebrae and, in the case of the PNS, through vagus nerve 30.

Potential choices of appropriate signals may be frequency critical as has been set forth by Sandblom and George, “Frequency Response in Resonance Behavior of Ionic Channel Currents Modulated by AC Fields” 1993, who indicate that ionic channel currents calculated are frequency-dependent, describing the rates of transports of ions through channels. “Liboff, et al, has proposed an optimum fluctuating magnetic field frequency for regulating transport frequency regulating transport across ionic membrane. See U.S. Pat. No. 5,160,591 (1992). The molecular characterization of the neuronal calcium channel has been studied by Perez-Ryes. Nature 1998, 391:896.

It is anticipated that, in one embodiment, appropriate electrical magnetic or electro-magnetic stimulation can be furnished to the T6 to T12 vertebrae by the use of probes, and that these would include both low and high frequency fields, inclusive of. AC and DC, with AC upon a DC carrier or, as taught by Liboff above, using a Helmholz Coil to produce cyclotronic magnetic fields that are imparted to tissue or nerves of interest.

Recent developments in molecular cell biology have confirmed the principles reflected in FIGS. 2-6 above. For example, Jiang et al, Rockfeller University, 2002, states: Ion channels exhibit two essential biophysical properties: a) selective ion conduction, and b) the ability to gate-open in response to an appropriate stimulus. Two general categories of ion channel gating are defined by the initiating stimulus: ligand binding (neurotransmitter—or second-messenger-gated channels) or membrane voltage (voltage-gated channels), per FIGS. 4-6. The structural basis of ligand gating in a K+ channel is that it opens in response to intracellular Ca2⁺. Jiang author reports he has they cloned, expressed, and analysed electrical properties, and determined the crystal structure of a K+ channel from methanobacterium thermoautotrophicum in the (Ca2+) bound, opened state and that eight RCK domains (regulators of K+ conductance) form a gating ring at the intracellular membrane surface. The gating ring uses the free energy of Ca2+ binding to perform mechanical work to open the pore.

Many forms of cellular dysfunction have been related to the electrical call to action of cells upon sensing of the voltage gradient, the cell membrane required to open the ionic channels. As such, electrical signals are modulated by the flow of calcium anions from and to the external medium thus affecting intra-cellular storage. Correction of any malfunction in the ability of the cell to provide a proper signal is summarized in FIG. 1 and shown schematically in FIG. 2. The present invention thereby provides necessary currents and voltages, as summarized in FIGS. 3-6, and taught in my Application Serial No. 13/065,015 necessary to optimize the flow of calcium anions to thereby restore normal function of dysfunctional cells within a given tissue. It is to be appreciated that other anions and their channels, e.g., potassium or sodium channels, may be associated with a given dysfunction.

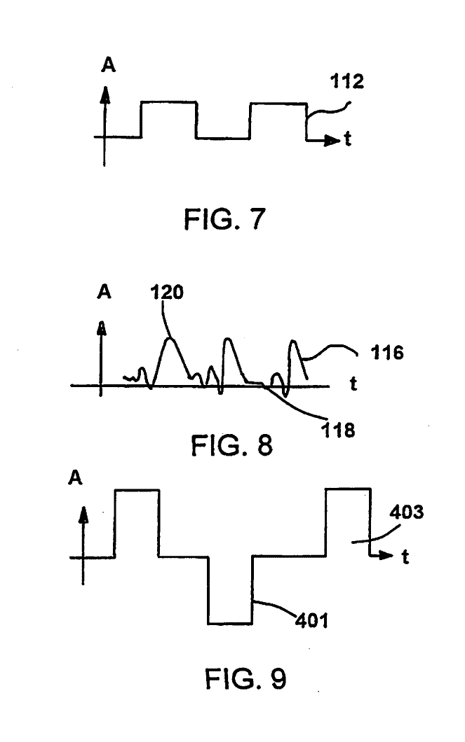

Shown in FIG. 7 is a waveform of a type used during initial probe emission 112, that is, when searching for a source of dysfunction. FIG. 8 shows a waveform that is received when a source of dysfunction is located responsive to waveform of an initial probe emission. The waveform typical of the type used at the start of treatment indicates a cell health positive response 112. However, 116 and 118 are health negative responses. See FIG. 8. The waveform of FIG. 9 is an algorithm simplified version of the waveform of FIG. 8. It includes a lower portion 401 (health negative) and upper portion 403 (health positive) which, it is to be appreciated, may be adapted in shape, dependent upon the needs of a technician to better locate somatic treatment points, such as area 403.

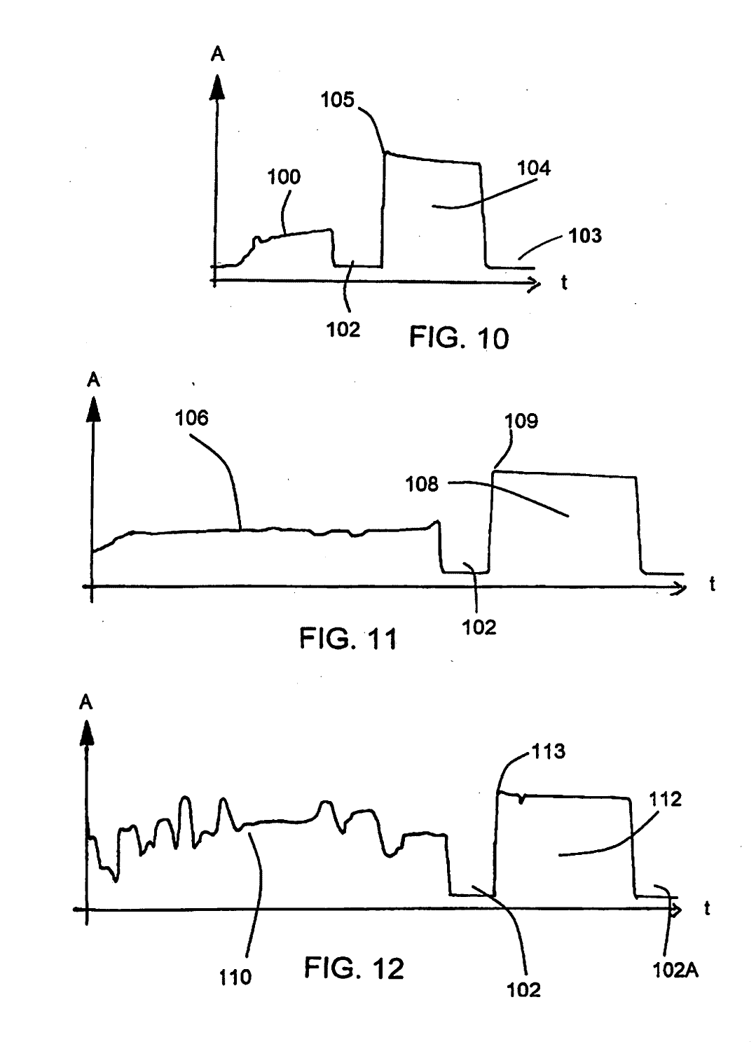

FIG. 10 is a waveform of an initial responsive following the beginning of treatment at a target site. Shown is the amplitude of a weaker segment 100 of the responsive wave, followed by transition 102 to a second segment 104 of the responsive waveform, which is a stronger or healthier response, which is followed by a further transition 103 at the right of FIG. 10. Edge 105 of waveform 104 is indicative of a higher capacitance of the part of the cell of the target site.

FIG. 11 is a view, sequential to that of FIG. 10, showing the result of initial treatment at a first site. Therein is shown that the amplitude of segment and shape of segment 100 of FIG. 10 has now increased to segment 106 of FIG. 11. This increased height waveform, as well as increased uniformity of the geometry of the waveform 106 is indicative of an induced healing process. Further is an area in which the portion 104 of FIG. 10 has changed to segment 108 shown in FIG. 11. Both segments 106 and 108 are indicative of a greater duration and length which correlates to healing at the site. Also shown is edge 109. The reduction in sharpness of edge 109 of segment 108 of the waveform indicates healing relative to the edge 105 in segment 104 of the waveform of FIG. 10.

FIG. 12 is a view at a second locus treatment of the spine showing that the treatment site exhibits a static-like and irregular segment 110 followed by a stronger segment 112 exhibiting a higher capacitance area 113. At 102 is shown a transition between segments.

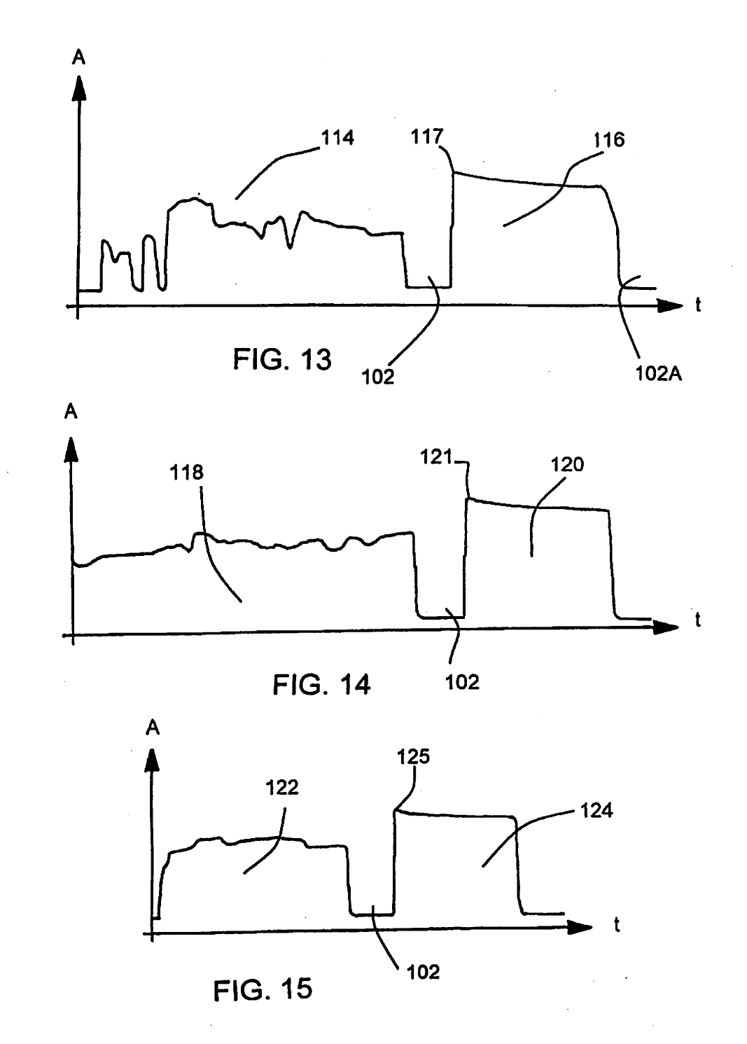

FIG. 13 is another view of the second locus of treatment within the same general therapy area. A similar pattern of static followed by a healthier area 116 is observed both upon waveforms and in an audio transform thereof (static sound versus a smooth sound). The treatment probe is moved slightly until an area of malfunction appears visually as a weak signal and, in audio, as a static or screeching sound. After a period of application of complex EM wave and energy patterns, a more positive response may be seen in FIG. 14 as much healthier segments 118 and 120, with capacitative edge 121 upon segment 120.

FIG. 15 is a waveform sequential to that of FIG. 14 in which segment 118 of FIG. 14 may be seen to be slightly changed into waveforms 122 and 124. However, segment 118 of FIG. 14 has now strengthened into a healthier waveform segment 122. Note greater the height of segment 122 versus 118. Pointed edge 125 shown in FIG. 15, is indicative of rate of change of capacitance at a treatment site, which is not desirable. Thus the waveform of FIG. 15 shows general strengthening with, however, a loss in length of the segment and a sharper edge 125 to waveform 124. Repetative treatments of about ten minutes are needed to maximize all parameters.

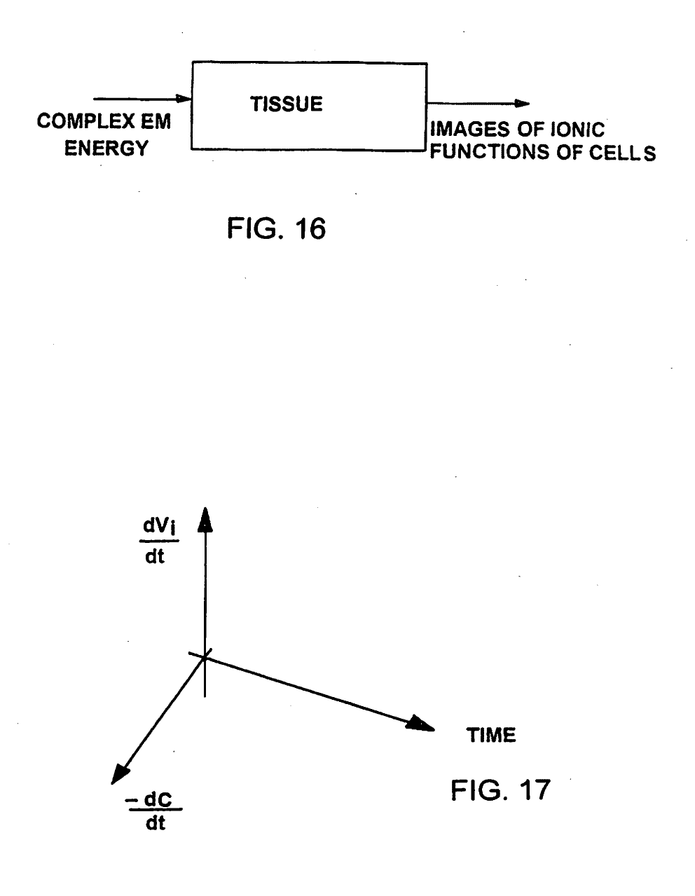

FIG. 16 is a block diagrammatic view showing how, by the input of a complex electrical and magnetic signals to a tissue site of interest, a three-dimensional image based upon a map of any selectable two of the parameters (versus time) may be accomplished, including signal stability or rate of change in amplitude of signals. One may also calculate the first or second derivative of absolute signal amplitude as a more precise measure of signal stability. Capacitance is a further parameter that may be mapped against time to show how the effects of the treatment signal are retained at the treatment site. The derivative of capacitance may be mapped to show the rate of discharge of capacitance. Also, voltage across the cell membrane at the treatment site may, as in the view of FIGS. 4-6, be used as an important parameter, in combination with others, to produce two or three dimensional imaging of value to the treating technician and physician. The rate of change of voltage across cell membrane is also an important parameter which may be mapped both to provide a more complete picture of a user dysfunction and the result which the present therapy is effecting during treatment and between treatment session. An example of useful parameters which may be mapped in three-dimensions is shown in FIG. 17.

Data showing the effect of the present therapy is as follows:
Blood sugar before treatment 320
After one hour 302
After two hours 258

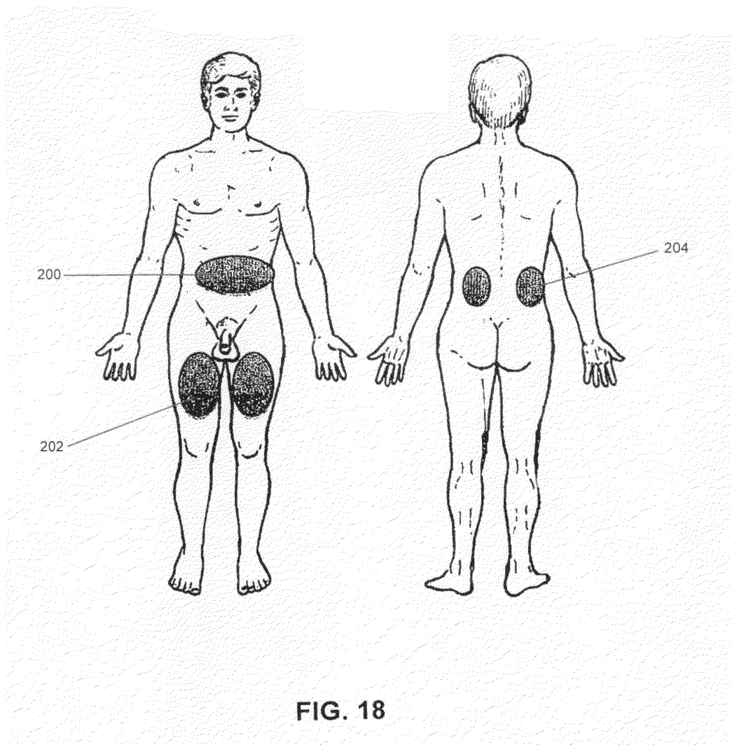

Shown in FIG. 18 are illustrations of the manner in which the above-described electrical simulations to the spine may be effected through the use of probe-embedded pads or patches selectably applied to the pancreas, liver and large quad muscles for the treatment of diabetes, and pads applied to the kidneys for purposes of treatment of the kidneys, and pads applied to the lower back near T4 for relief of hypertension.

From the above, the instant invention may be practiced through the use of an EMF pad or probe assembly for the treatment and recognition of abnormalities of nerves and other cells and tissues of the human body including membrane flow of ions of cells associated with such conditions. Such an assembly includes a probe; at least a ferro-magnetic core positioned within said probe or pad; and at least one induction coil wound about at least one core. An assembly will typically include a plurality of probes and a corresponding plurality of coils thereabout in which at least one of said cores defines a sphere integral to a core at a distal end. An electrical pulse train is furnished to a proximal end of at least one of said coils wherein a pulsed magnetic wave is thereby provided along an axis of said cores to the distal ends thereof. Such electrical pulse train therefore generates pulsed magnetic fields axial to said cores and extending as magnetic outputs from the distal ends of the probes. More than one, and preferably two probes are used concurrently such that two geometries of pulsed magnetic fields are emitted from sides or distal ends thereof. Typically one of such probes would be the above-described probe having a spherical end while the other probe would be a non-spherical probe. As may be appreciated, the use of said sphere is useful in generating magnetic field outputs of the probes having a hemispherical geometry.

In accordance with the medical principles of treatment discussed above, the pulsed magnetic field output of the probes is preferably of an opposing electron-magnetic polarity to that generated by abnormal tissue to be treated. Thus provided is a means for generating a pulsed electromagnetic field, at a distal end of the at least one of said probes, having a countervailing electro-magnetic geometry to that generated by an abnormal flow of electrons across said cell membranes of a given tissue.

The invention, as above described, also includes an audio transform for expressing electro-magnetic changes and responses of abnormal cells and tissues into human audible frequencies. Using such frequencies, one may adjust the magnitude and geometry of the above-described electro-magnetic field outputs of the probes. Audio software recognition, as well as clinical training of technicians, enables one to recognize the meaning of the human audible frequency outputs as correlating to desirable or undesirable voltage gradients shown in FIGS. 7-15 across cell membranes of cells of an afflicted tissue. Per FIG. 17, visual means may, similarly, be provided for the viewing of the reactive parameters of the countervailing electro-magnetic geometric provided in the present therapy and by the afflicted tissue.

Accordingly, while there has been shown and described the preferred embodiment of the invention is to be appreciated that the invention may be embodied otherwise than is herein specifically shown and described and, within said embodiment, certain changes may be made in the form and arrangement of the parts without departing from the underlying ideas or principles of this invention, as claimed herein.

1. An EMF probe assembly for the stimulation of T6 through T12 vertebrae and related neural offshoots, to treat diabetic symptoms, the assembly comprising:
(a) a probe;
(b) at least one core formed of a metallic material positioned within said probe; and
(c) at least one induction coil wound around said at least one core

2. The assembly as recited in claim 1, comprising a plurality of probes and a corresponding plurality of cores and coils thereabout in which at least one of said cores defines a sphere integral to a core at a distal end of the probe.

3. The assembly as recited in claim 2, further comprising:
an electrical pulse train furnished to a proximal end of at least one of said coils wherein a pulsed magnetic wave is thereby provided along an axis of said cores to distal ends thereof.

4. The assembly as recited in claim 3, further comprising:
a pulsed magnetic field at a distal end of said probe by furnishing an electrical current to said proximal end of said at least one coil.

5. The assembly as recited in claim 3, in which said electrical pulse train generates pulsed magnetic fields from coil at said distal end of at least one of said probes.

6. The assembly as recited in claim 5, comprising:
means for simultaneously emitting pulsed magnetic fields from said distal end of two probes.

7. The assembly as recited in claim 5, comprising:
means for simultaneously emitting a pulsed magnetic field from said spherical probe end and from one non-spherical probe end of another probe.

8. The assembly as recited in claim 7 in which a induction coils comprise:
means for generating axial fields and in combination with said sphere of one probe, hemispherical fields.

9. The assembly as recited in claim 5, comprising:
means for generating a pulsed magnetic field of opposing magnetic polarity to that generated by abnormal tissue to be treated.

10. The assembly as recited in claim 5, comprising:
a pulsed electro-magnetic field, at said distal end of said distal end of at least one of said probes, having a countervailing electro-magnetic geometry to that generated by an abnormal flow of ions across a cell membrane of a given tissue.

11. The assembly as recited in claim 10, further comprising:
an audio transform for expressing electro-magnetic changes and responses of abnormal cells and tissues into human audible frequencies.

12. The assembly as recited in claim 11, further comprising:
means for adjusting said pulsed electro-magnetic fields in response to said audible frequencies.

13. The assembly as recited in claim 11, in which said audio transform comprises:
means for recognition of said responses of abnormal coils as a function of undesirable voltage gradient across membranes of cells of an affected tissue.

14. The assembly as recited in claim 12, in which said audio transform comprises:
means for recognition of said responses of abnormal coils as a function of undesirable voltage gradient across cell membrane of cells of an affected tissue.

15. The assembly as recited in claim 10, further comprising:
means for adjusting said electro-magnetic fields in response to an EM field spectrograph of a tissue abnormality.

16. The assembly as recited in claim 10, comprising:
means for viewing reactive parameters of said countervailing electromagnetic geometry.

17. The assembly as recited in claim 1, embedded within a pad or patch for contact with or near vertebrae T6 through T12 or their neural offshoots
